(19) United States
(12) Patent Application Publication
(10) Pub. No.: US 2009/0252654 A1
(43) Pub. Date: Oct. 8, 2009

(54) AIR CLEANER

(75) Inventors: CHIH-PENG HSU, HuKou (TW); CHUNG-MIN CHANG, HuKou (TW); TSE-AN LEE, HuKou (TW)

Correspondence Address: PCE INDUSTRY, INC.;ATT. Steven Reiss, 288 SOUTH MAYO AVENUE, CITY OF INDUSTRY, CA, 91789, US

(73) Assignee: ADVANCED OPTOELECTRONIC TECHNOLOGY, INC., Hsinchu Hsien (TW)

(21) Appl. No.: 12/240,422

(22) Filed: Sep. 29, 2008

(30) Foreign Application Priority Data
Apr. 2, 2008 (CN) 200810300815.6

(51) Int. Cl. A61L 9/00

(52) U.S. Cl. 422/122

(57) ABSTRACT

An air cleaner includes a photo-catalyst filter, a light source and an ozone generator. The light source is configured for emitting light having a given wavelength to activate the photo-catalyst layer to decompose contaminants thereon. The ozone generator is configured for generating ozone flowing through the photo-catalyst layer to promote decomposition of the contaminants on the photo-catalyst layer.

### CROSS-REFERENCE TO RELATED APPLICATIONS

This application is related to the following commonly-assigned copending applications: Ser. No. ______, entitled “PHOTO-CATALYST AIR CLEANER” (attorney docket number US 18919). Disclosures of the above-identified application is incorporated herein by reference.

### BACKGROUND

1. Field of the Invention

The present invention relates to air cleaners, and particularly to an air cleaner with an ozone generator.

2. Description of Related Art

In recent years, with awareness of the dangers of air pollution, air cleaners are more widely used to improve air quality of the environment.

Most conventional air cleaners use ultraviolet light (UV light) to excite a photo-catalyst, the excited photo-catalyst can then decompose contaminants and sterilize microbes in the air, thus cleaning air is achieved. However, many kinds of contaminants that cannot be decomposed immediately may be deposited on the photo-catalyst, thus preventing UV emissions efficiently exciting the photo-catalyst, and degrading air cleaning properties of the air cleaner.

What is needed, therefore, is an improved air cleaner which can overcome the above shortcomings.

### SUMMARY

An air cleaner includes a photo-catalyst filter, a light source and an ozone generator. The light source is configured for emitting light having a given wavelength to activate the photo-catalyst layer to decompose contaminants thereon. The ozone generator is configured for generating ozone flowing through the photo-catalyst layer to promote decomposition of the contaminants on the photo-catalyst layer.

Other advantages and novel features of the present air cleaner will become more apparent from the following detailed description when taken in conjunction with the accompanying drawings, in which:

### BRIEF DESCRIPTION OF THE DRAWINGS

Many aspects of the present air cleaner can be better understood with reference to the following drawings. The components in the drawings are not necessarily drawn to scale, the emphasis instead being placed upon clearly illustrating the principles of the present illumination device. Moreover, in the drawings, like reference numerals designate corresponding parts throughout the several views.

FIG. 1 is a cross-sectional view of an air cleaner, in accordance with a first exemplary embodiment.

FIG. 2 is a schematic view of a photo-catalyst filter of FIG. 1.

FIG. 3 is a partial cross-sectional and amplified view of the photo-catalyst filter of FIG. 2 taken along a direction shown by section line III-III.

FIG. 4 is a cross-sectional view of an air cleaner, in accordance with a second exemplary embodiment.

### DETAILED DESCRIPTION

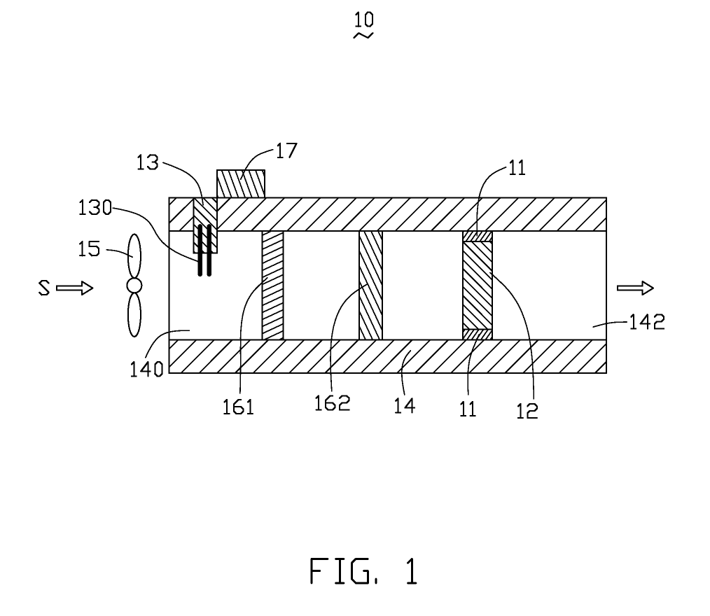

Referring to FIG. 1, an air cleaner 10, in accordance with a first embodiment, comprises a light source 11, a photo-catalyst filter 12, and an ozone generator 13.

The light source 11 can be an ultraviolet (UV) lamp, such as a UV fluorescent lamp, which is able to emit UV light. Preferably, the light source 11 is a UV light emitting diode. Additionally, there can be more than one light source 11.

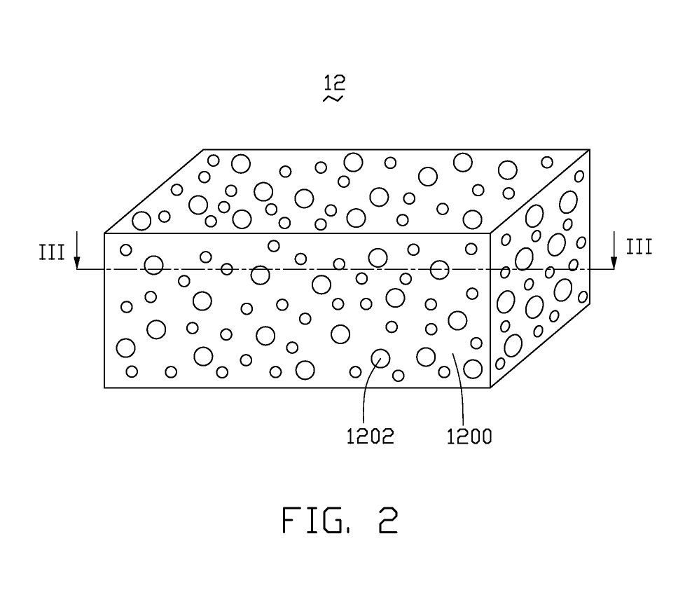

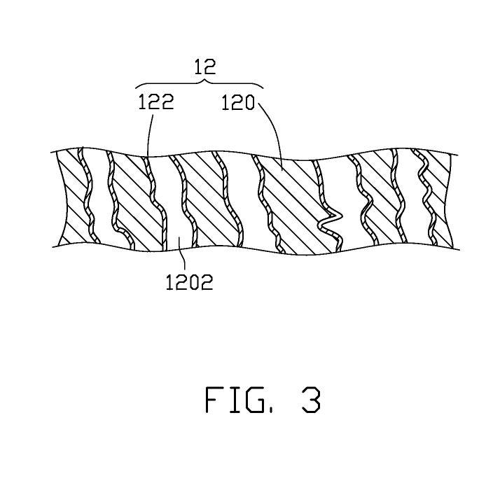

Referring to FIGS. 2-3, the photo-catalyst filter 12 comprises a substrate 120 and a photo-catalyst layer 122. The substrate 120 has a surface 1200, the photo-catalyst layer 122 is coated on the surface 1200 of the substrate 120 by a sintering process, and configured for contact with air for decomposing contaminants and sterilizing microbes contained in the air. The substrate 120 has a porous structure. That is, the substrate 120 has a plurality of holes 1202 defined on the surface 1200 of the substrate 120. The holes 1202 may interpenetrate or be separated from each other. The photo-catalyst layer 122 can be further coated on internal walls of the holes, thus increasing surface area of the photo-catalyst layer 122, to improve decomposing and sterilizing capability of the photo-catalyst filter 12. Generally, the substrate 120 can be made of ceramic, aluminum or nickel. The photo-catalyst layer 122 can be made of nanometer sized photo-catalyst material, such as titanium dioxide (TiO₂), tin oxide (SnO₂), zinc oxide (ZnO), tungsten oxide (WO₃), iron oxide (Fe₂O₃), SeTiO₃, cadmium selenide (CdSe), KTaO₃, cadmium sulfide (CdS) or niobium oxide (Nb₂O₅).

The ozone generator 13 includes surface discharge electrodes 130 for generating high voltages, to ionize oxygen to form ozone with higher oxidative ability (O+O₂→O₃).

The air cleaner 10 further comprises a chamber 14. The chamber 14 includes an inlet 140 and an outlet 142. The ozone generator 13, the photo-catalyst filter 12, and the light source 11 are arranged in the chamber 14 between the inlet 140 and the outlet 142. Additionally, an airflow channel (shown by the arrowhead S in FIG. 1) is defined from the inlet 140 to the outlet 142.

The air cleaner 10 can further comprise an air driving member 15, such as a fan. The fan is disposed in the airflow channel. Upon driving the fan, external air containing contaminants, such as dust and the like is drawn through the inlet 140 into the chamber 14, the contaminants then pass through the airflow channel and are discharged to outside of the chamber 14 through the outlet 142. And, particularly, the flowing of the ozone is promoted from the inlet 140 to the outlet 142. In general, a primary filter 161, is placed in the airflow channel in front of the photo-catalyst filter 12, and is used for primary filtering of airborne particles bigger than 5 micrometers (μm) in diameter, and a high efficiency particulate air filter (HEPA) 162, is placed between the primary filter 161 and the photo-catalyst filter 12, and is used to remove most of (at least 99.97%) airborne particles 0.3 micrometers (μm) in diameter or more.

In operation, the photo-catalyst layer 122 is exposed to the emission of the UV light emitting diode to absorb the UV light, and electron-hole pairs are excited from within the photo-catalyst layer 122 to a surface thereof, to initiate reduction/oxidation reactions (redox) with organic contaminants adsorbed on the surface. Therefore, the organic contaminants can be oxidized, achieving decomposition of the contaminants, and sterilization of microbes.

In conventional filters, contaminants that cannot be decomposed immediately may be deposited on the photo-catalyst layer 122, thus preventing emission of the UV light. To prevent such build up on the photo-catalyst layer 122, the ozone generator 13 generates ozone with higher oxidative ability to flow through the photo-catalyst layer 122, to promote decomposition of the contaminants thereon.

The ozone generator 13 can be disposed at one side of the filter 161 away from the filter 162, or between the filters 161, 162, or in front of the filters 161, 162. Provided ozone from the ozone generator 13 is able to pass through the photo-catalyst layer 122, placement of the ozone generator 13 is arbitrary.

Because excess ozone may be harmful to health of people, a controller 17 can be applied to limit ozone generation by the ozone generator 13. The controller 17 can be a clock generator, which is able to control the ozone generator 13 to generate ozone intermittently.

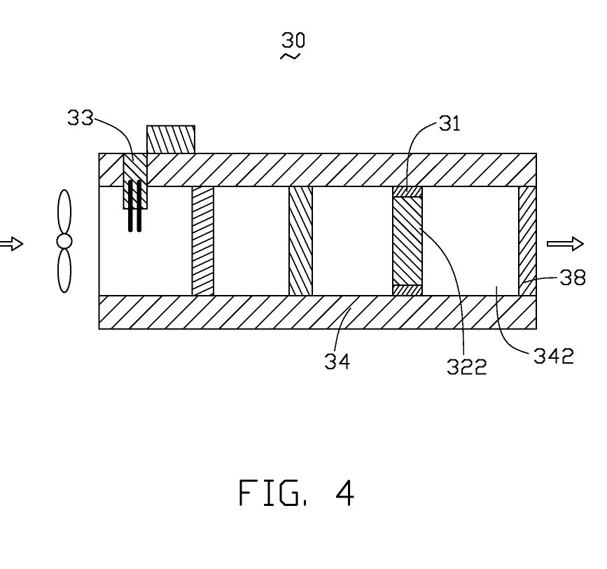

FIG. 4 shows an air cleaner 30, in accordance with a second embodiment. The air cleaner 30 is distinguished from the air cleaner 10 in that a humidifier 38 is included. The humidifier 38 is configured for generating water vapor to reduce the volume of ozone generated and expelled by the air cleaner 30.

The humidifier 38 is placed at the outlet 342 of the chamber 34. The ozone passing through the photo-catalyst layer 322 is dissolved in the water vapor before discharging to the outside of the chamber 34 through the outlet 342. Then the water vapor is discharged harmlessly outside of the chamber 34 through the outlet 342.

It is believed that the present invention and its advantages will be understood from the foregoing description, and it will be apparent that various changes may be made thereto without departing from the spirit and scope of the invention or sacrificing all of its material advantages, the examples hereinbefore described merely being preferred or exemplary embodiments of the invention.

What is claimed is:

1. An air cleaner, comprising
a photo-catalyst filter comprising a photo-catalyst layer;
a light source for emitting light having a given wavelength to activate the photo-catalyst layer to decompose contaminants thereon;
an ozone generator for generating ozone flowing through the photo-catalyst layer to promote decomposition of the contaminants on the photo-catalyst layer.

2. The air cleaner of claim 1, wherein the photo-catalyst filter further comprises a substrate, the substrate has a surface, and the photo-catalyst layer is formed on the surface.

3. The air cleaner of claim 2, wherein the substrate has a porous structure with a plurality of holes, and the photo-catalyst layer is formed on internal walls of the holes.

4. The air cleaner of claim 2, wherein the substrate is comprised of a material selected from the group consisting of: ceramic, aluminum and nickel.

5. The air cleaner of claim 1, further comprising a chamber, the chamber comprising an inlet and an outlet, the ozone generator, the photo-catalyst filter and the light source arranged in the chamber between the inlet and the outlet, an air flowing channel defined in the chamber between the inlet and the outlet.

6. The air cleaner of claim 5, further comprising an air driving member for promoting flowing of the ozone in the channel from the inlet to the outlet.

7. The air cleaner of claim 5, wherein the air driving member includes a fan.

8. The air cleaner of claim 5, further comprising a humidifier, the humidifier placed at the outlet of the chamber for generating water vapor.

9. The air cleaner of claim 1, wherein the photo-catalyst layer is comprised of a material selected from the group consisting of: TiO₂, SnO₂, ZnO, WO₃, Fe₂O₃, SeTiO₃, CdSe, KTaO₃, CdS and Nb₂O₅.

10. The air cleaner of claim 1, wherein the light source is an ultraviolet light emitting diode.
